(19) United States
(12) Patent Application Publication
(10) Pub. No.: US 2025/0049340 A1
(43) Pub. Date: Feb. 13, 2025

(54) ELECTRONIC DEVICE AND METHOD FOR EVALUATING AUTOREGULATORY PATTERN OF CEREBRAL BLOOD FLOW

(71) Applicant: Han-Hwa Hu, Taipei City (TW)

(72) Inventors: Han-Hwa Hu, Taipei City (TW); Wen-Yung Sheng, Taipei City (TW); Hung-Yi Hsu, Taichung City (TW); Ke Han, Shenzhen (CN); Cheng-Yen Wang, Taoyuan City (TW)

(73) Assignee: Han-Hwa Hu, Taipei City (TW)

(21) Appl. No.: 18/429,462

(22) Filed: Feb. 1, 2024

(30) Foreign Application Priority Data
Aug. 8, 2023 (TW) 112129815

(51) Int. Cl. A61B 5/0285; A61B 5/00; A61B 5/021; G16H 30/40

(52) U.S. Cl. CPC A61B 5/0285; A61B 5/021; A61B 5/4064; A61B 5/746; G16H 30/40

(57) ABSTRACT

An electronic device and a method for evaluating an autoregulatory pattern of cerebral blood flow are provided. The method includes the following. Each data point of a first data set is grouped according to a plurality of blood pressure ranges to generate a plurality of data groups respectively corresponding to the plurality of blood pressure ranges. A plurality of average values of blood flow velocity of the plurality of data groups are calculated. A first linear regression operation is performed on the plurality of average values of blood flow velocity to generate a first regression line. A first slope of the first regression line is calculated to obtain a first indicator. A plurality of data sets including the first data set are grouped according to the first indicator to generate a plurality of autoregulatory pattern groups. It is determined that a second data set corresponds to one of the plurality of autoregulatory pattern groups. An autoregulatory pattern of the second data set is output.

### CROSS-REFERENCE TO RELATED APPLICATION

This application claims the priority benefit of Taiwan application serial no. 112129815, filed on Aug. 8, 2023. The entirety of the above-mentioned patent application is hereby incorporated by reference herein and made a part of this specification.

### BACKGROUND

### Technical Field

The disclosure relates to an electronic device and a method for evaluating an autoregulatory pattern of cerebral blood flow.

### Description of Related Art

When the blood pressure of the human body changes, the autoregulatory mechanism of the cerebral blood flow can adjust the caliber of cerebral blood vessels to generate cerebral perfusion pressure (CPP) so that the cerebral blood flow (CBF) can be maintained in a fixed range. At present, many studies have proposed methods to predict the occurrence of cerebrovascular disease by analysis of the cerebral blood flow. However, the autoregulatory pattern of cerebral blood flow in different patients can be different, and existing medical equipment cannot define the autoregulatory pattern of cerebral blood flow in patients. In this way, if the autoregulatory pattern of cerebral blood flow of the patient cannot be used, the prediction of the various cerebrovascular disorders by analysis of cerebral blood flow of a patient can be distorted.

### SUMMARY

The disclosure provides an electronic device and a method for evaluating an autoregulatory pattern of cerebral blood flow, which can be used to judge the autoregulatory pattern of cerebral blood flow of a patient.

An electronic device for evaluating an autoregulatory pattern of cerebral blood flow of the disclosure includes a processor and a transceiver. The processor is coupled to the transceiver and is configured to: receive a first data set through the transceiver, wherein a data point in the first data set includes blood pressure and blood flow velocity corresponding to the blood pressure; group each data point in the first data set according to a plurality of blood pressure ranges to generate a plurality of data groups respectively corresponding to the plurality of blood pressure ranges; calculate a plurality of average values of the blood flow velocity respectively corresponding to the plurality of data groups; perform a first linear regression operation on the plurality of average values of the blood flow velocity to generate a first regression line; calculate a first slope of the first regression line to obtain a first indicator; group a plurality of data sets including the first data set according to the first indicator to generate a plurality of autoregulatory pattern groups; receive a second data set through the transceiver, and determine that the second data set corresponds to one of the plurality of autoregulatory pattern groups; and output an autoregulatory pattern of the second data set, wherein the autoregulatory pattern corresponds to one of the plurality of autoregulatory pattern groups.

In an embodiment of the disclosure, the above-mentioned processor is further configured to: calculate cerebrovascular resistance according to the blood pressure and the blood flow velocity; calculate a plurality of average values of the cerebrovascular resistance respectively corresponding to the plurality of data groups; perform a second linear regression operation on the plurality of average values of the cerebrovascular resistance to generate a second regression line; calculate a second slope of the second regression line to obtain a second indicator; and group the plurality of data sets according to the first indicator and the second indicator to generate the plurality of autoregulatory pattern groups.

In an embodiment of the disclosure, the above-mentioned processor is further configured to: calculate a plurality of standard deviations of the blood flow velocity respectively corresponding to the plurality of average values of the blood flow velocity; generate an image according to the plurality of average values of the blood flow velocity and the plurality of standard deviations of the blood flow velocity; and output the image.

In an embodiment of the disclosure, the above-mentioned processor is further configured to: communicatively connect to an ultrasonic instrument through the transceiver, and receive the blood flow velocity of the data point from the ultrasonic instrument.

In an embodiment of the disclosure, the above-mentioned processor is further configured to: communicatively connect to a sphygmomanometer through the transceiver, and receive the blood pressure of the data point from the sphygmomanometer.

In an embodiment of the disclosure, each data point in the above-mentioned first data set corresponds to each heartbeat of a subject.

In an embodiment of the disclosure, the above-mentioned processor is further configured to: perform an interpolation operation on the first data set to up-sample the first data set.

In an embodiment of the disclosure, the above-mentioned processor is further configured to: output an alert message in response to determining that the second data set corresponds to one of the plurality of autoregulatory pattern groups.

A method for evaluating an autoregulatory pattern of cerebral blood flow of the disclosure includes the following. A first data set is received, and a data point in the first data set includes blood pressure and blood flow velocity corresponding to the blood pressure. Each data point in the first data set is grouped according to a plurality of blood pressure ranges to generate a plurality of data groups respectively corresponding to the plurality of blood pressure ranges. A plurality of average values of the blood flow velocity respectively corresponding to the plurality of data groups are calculated. A first linear regression operation is performed on the plurality of average values of the blood flow velocity to generate a first regression line. A first slope of the first regression line is calculated to obtain a first indicator. A plurality of data sets including the first data set is grouped according to a first indicator to generate a plurality of autoregulatory pattern groups. A second data set is received, and it is determined that the second data set corresponds to one of the plurality of autoregulatory pattern groups. An autoregulatory pattern of the second data set is output, and the autoregulatory pattern corresponds to one of the plurality of autoregulatory pattern groups.

Based on the above, the electronic device of the disclosure can evaluate the autoregulatory pattern of cerebral blood flow of the patient according to the data point including the blood pressure and the blood flow velocity.

### BRIEF DESCRIPTION OF THE DRAWINGS

FIG. 1 is a schematic diagram of an electronic device for evaluating an autoregulatory pattern of cerebral blood flow according to an embodiment of the disclosure.

FIG. 2 is a schematic diagram of a regression line according to an embodiment of the disclosure.

FIG. 3 is a schematic diagram of a regression line according to an embodiment of the disclosure.

FIG. 4 is a schematic diagram of a plurality of autoregulatory pattern groups according to an embodiment of the disclosure.

FIG. 5 is a flowchart of a method for evaluating an autoregulatory pattern of cerebral blood flow according to an embodiment of the disclosure.

### DESCRIPTION OF THE EMBODIMENTS

In order to make the content of the disclosure more comprehensible to understand, embodiments are described below as examples according to which the disclosure can indeed be implemented. In addition, wherever possible, elements/components/steps with the same referential numbers in the drawings and embodiments represent the same or similar parts.

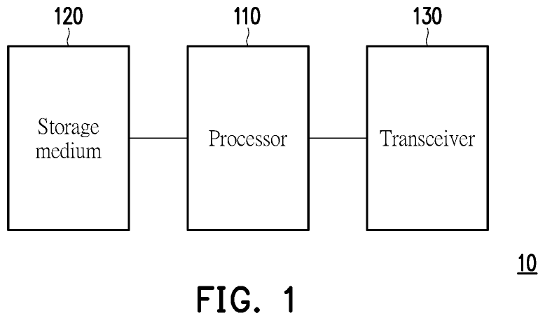

FIG. 1 is a schematic diagram of an electronic device 10 for evaluating an autoregulatory pattern of cerebral blood flow according to an embodiment of the disclosure. The electronic device 10 can include a processor 110, a storage medium 120, and a transceiver 130.

The processor 110 is, for example, a central processing unit (CPU), or other programmable general-purpose or special-purpose micro control units (MCUs), a microprocessor, a digital signal processor (DSP), a programmable controller, an application specific integrated circuit (ASIC), a graphics processing unit (GPU), an image signal processor (ISP)), an image processing unit (IPU), an arithmetic logic unit (ALU), a complex programmable logic device (CPLD), a field programmable gate array (FPGA), or other similar elements, or a combination of the above elements. The processor 110 can be coupled to the storage medium 120 and the transceiver 130, and access and execute a plurality of modules and various application programs stored in the storage medium 120.

The storage medium 120 is, for example, any form of fixed or movable random access memory (RAM), a read-only memory (ROM), a flash memory, a hard disk drive (HDD), a solid state drive (SSD), or a similar element, or a combination of the above elements, and is used to store the plurality of modules or the various application programs executable by the processor 110.

The transceiver 130 transmits and receives signals in a wireless or wired manner. The transceiver 130 can also perform operations such as low noise amplification, impedance matching, frequency mixing, up or down frequency conversion, filtering, amplification, and the like.

The processor 110 can receive a historical data set including a plurality of historical data points through the transceiver 130. Each historical data point can include information such as blood pressure and blood flow velocity corresponding to the blood pressure. In an embodiment, the processor 110 can communicatively connect to the ultrasonic instrument through the transceiver 130 and receive the blood flow velocity of the historical data points from the ultrasonic instrument. In an embodiment, the processor 110 can communicatively connect to the sphygmomanometer through the transceiver 130 and receive the blood pressure of the historical data points from the sphygmomanometer. Each historical data point in the historical data set can correspond to each heartbeat of a subject. For example, when the heartbeat of the subject occurs, the ultrasonic instrument and the sphygmomanometer can respectively define the blood flow velocity and blood pressure corresponding to the heartbeat to generate the historical data point. The processor 110 can receive the historical data set including the plurality of historical data points respectively corresponding to multiple heartbeats through the transceiver 130.

In an embodiment, the processor 110 can further perform an interpolation operation on the historical data set to up-sample the historical data set so as to increase the number of the historical data points in the historical data set.

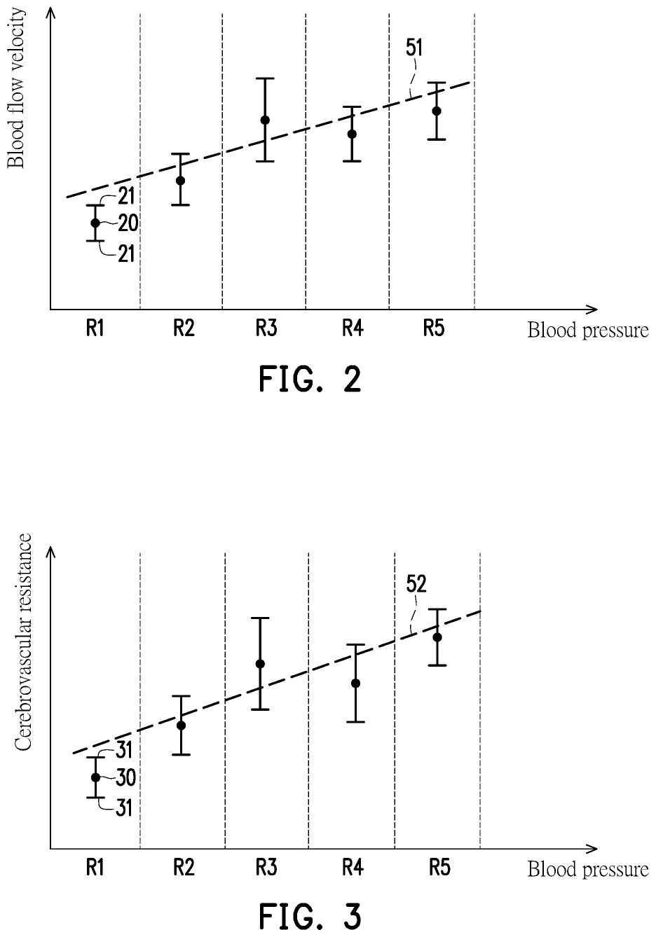

The processor 110 can group each historical data point in the historical data set according to a plurality of blood pressure ranges to generate a plurality of data groups respectively corresponding to the plurality of blood pressure ranges. The processor 110 can further calculate a plurality of average values of the blood flow velocity respectively corresponding to the plurality of data groups. FIG. 2 is a schematic diagram of a regression line 51 according to an embodiment of the disclosure. After obtaining the historical data set including the plurality of historical data points, the processor 110 can group each historical data point according to the blood pressure of each historical data point to generate the plurality of data groups including data groups R1, R2, R3, R4, and R5. Taking the data group R1 as an example, the processor 110 can calculate the average value of the blood flow velocity of all historical data points in the data group R1 to generate an average value of blood flow velocity 20. Based on similar steps, the processor 110 can calculate a corresponding average value of the blood flow velocity for each of the data groups R1, R2, R3, R4, and R5.

In an embodiment, the processor 110 can calculate a standard deviation of the blood flow velocity corresponding to the average value of the blood flow velocity according to the blood flow velocity of each historical data point in the data group. The processor 110 can generate an image as shown in FIG. 2 according to the plurality of average values of the blood flow velocity and a plurality of standard deviations of the blood flow velocity respectively corresponding to the plurality of average values of the blood flow velocity, and output the image through the transceiver 130 for reference by the user of the electronic device 10. Taking the data group R1 as an example, the processor 110 can calculate a standard deviation of blood flow velocity 21 corresponding to the average value of blood flow velocity 20 according to the blood flow velocity of each historical data point in the data group R1. Based on similar steps, the processor 110 can calculate a corresponding standard deviation of the blood flow velocity for each of the data groups R1, R2, R3, R4, and R5. The processor 110 can generate an image as shown in FIG. 2 according to the average values of the blood flow velocity and the standard deviations of the blood flow velocity of the data groups R1, R2, R3, R4, and R5, and output the image for user's reference.

After obtaining the plurality of average values of the blood flow velocity respectively corresponding to the plurality of data groups, the processor 110 can perform a linear regression operation on the plurality of average values of the blood flow velocity to generate a regression line. The processor 110 can calculate the slope of the regression line to obtain a first indicator corresponding to the historical data set. Taking FIG. 2 as an example, the processor 110 can perform a linear regression operation on the plurality of average values of the blood flow velocity (including the average value of blood flow velocity 20 of the data group R1) to generate the regression line 51. The processor 110 can calculate a slope of the regression line 51 to obtain the first indicator corresponding to the historical data set.

On the other hand, the processor 110 can calculate a corresponding cerebrovascular resistance (CVR) for each historical data point in the historical data set according to formula (1), where CVR represents cerebrovascular resistance, ΔP represents blood pressure of a historical data point, and F represents blood flow velocity of a historical data point.

$$CVR = \frac{Δ P}{F} \qquad ( 1 )$$

Next, the processor 110 can calculate a plurality of average values of the cerebrovascular resistance respectively corresponding to the plurality of data groups. FIG. 3 is a schematic diagram of a regression line 52 according to an embodiment of the disclosure. After the processor 110 groups each historical data point according to the blood pressure of each historical data point to generate data groups R1, R2, R3, R4, and R5, taking the data group R1 as an example, the processor 110 can calculate the average value of the cerebrovascular resistance of all historical data points of the data group R1 to generate an average value of cerebrovascular resistance 30. Based on similar steps, the processor 110 can calculate a corresponding average value of the cerebrovascular resistance for each of the data groups R1, R2, R3, R4, and R5.

In an embodiment, the processor 110 can calculate a standard deviation of the cerebrovascular resistance corresponding to the average value of the cerebrovascular resistance according to the cerebrovascular resistance of each historical data point in the data group. The processor 110 can generate an image as shown in FIG. 3 according to the plurality of average values of the cerebrovascular resistance and the plurality of standard deviations of the cerebrovascular resistance respectively corresponding to the plurality of average values of the cerebrovascular resistance, and output the image through the transceiver 130 for reference by the user of the electronic device 10. Taking the data group R1 as an example, the processor 110 can calculate a standard deviation of cerebrovascular resistance 31 corresponding to the average value of cerebrovascular resistance 30 according to the cerebrovascular resistance of each historical data point in the data group R1. Based on similar steps, the processor 110 can calculate a corresponding standard deviation of the cerebrovascular resistance for each of the data groups R1, R2, R3, R4, and R5. The processor 110 can generate an image as shown in FIG. 3 according to the average value of the cerebrovascular resistance and the standard deviation of the cerebrovascular resistance of the data groups R1, R2, R3, R4, and R5, and output the image for user's reference.

After obtaining the plurality of average values of the cerebrovascular resistance respectively corresponding to the plurality of data groups, the processor 110 can perform a linear regression operation on the plurality of average values of the cerebrovascular resistance to generate a regression line. The processor 110 can calculate the slope of the regression line to obtain a second indicator corresponding to the historical data set. Taking FIG. 3 as an example, the processor 110 can perform a linear regression operation on the plurality of average values of the cerebrovascular resistance (including the average value of cerebrovascular resistance 30 of the data group R1) to generate the regression line 52. The processor 110 can calculate a slope of the regression line 52 to obtain the second indicator corresponding to the historical data set.

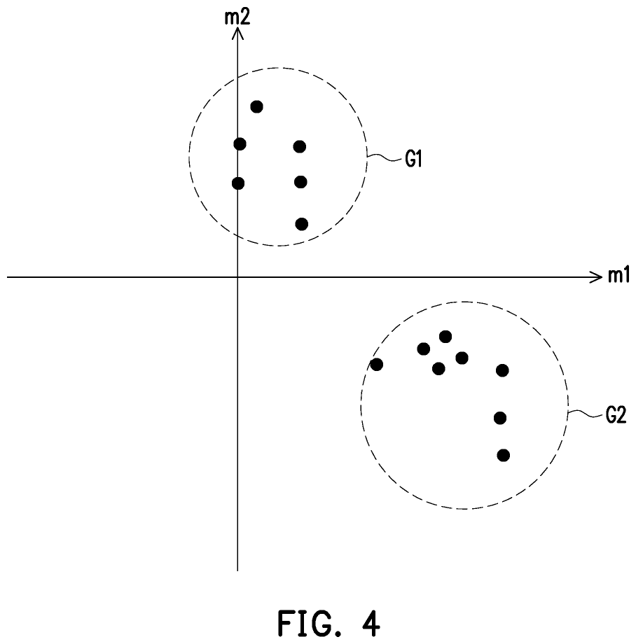

The processor 110 can calculate corresponding first indicator and second indicator for each historical data set in a plurality of historical data sets according to the above steps. Next, the processor 110 can group the plurality of historical data sets according to the first indicator and the second indicator of each historical data set to generate a plurality of autoregulatory pattern groups. The plurality of autoregulatory pattern groups can serve as models for evaluating autoregulatory patterns. FIG. 4 is a schematic diagram of a plurality of autoregulatory pattern groups according to an embodiment of the disclosure. Each point in FIG. 4 represents a historical data set, m1 represents a first indicator, and m2 represents a second indicator. The processor 110 can group the plurality of historical data sets into an autoregulatory pattern group G1 and an autoregulatory pattern group G2 according to the first indicator m1 and the second indicator m2 based on the grouping algorithm. The autoregulatory pattern group G1 and the autoregulatory pattern group G2 can serve as models for evaluating autoregulatory patterns.

Specifically, the processor 110 can receive a current data set including a plurality of current data points through the transceiver 130. Each current data point can include information such as blood pressure and blood flow velocity corresponding to the blood pressure. The processor 110 can group each current data point in the current data set according to a plurality of blood pressure ranges to generate a plurality of data groups respectively corresponding to the plurality of blood pressure ranges. The processor 110 can further calculate a plurality of average values of the blood flow velocity and a plurality of average values of cerebrovascular resistance respectively corresponding to the plurality of data groups. Then, the processor 110 can perform a linear regression operation on the plurality of average values of the blood flow velocity to generate a regression line, thereby generating a first indicator corresponding to the current data set. The processor 110 can also perform a linear regression operation on the plurality of average values of the cerebrovascular resistance to generate a regression line, thereby generating a second indicator corresponding to the current data set.

After obtaining the first indicator and the second indicator of the current data set, the processor 110 can determine which autoregulatory pattern group the current data set belongs to according to the first indicator and the second indicator of the current data set. The processor 110 can assign the current data set to a specific autoregulatory pattern group (e.g., autoregulatory pattern group G1) in a plurality of autoregulatory pattern groups (e.g., autoregulatory pattern groups G1 and G2) according to the first indicator and the second indicator of the current data set based on a grouping algorithm. The processor 110 can output the grouping result of the current data set (i.e., the above-mentioned specific autoregulatory pattern group) through the transceiver 130 for user's reference.

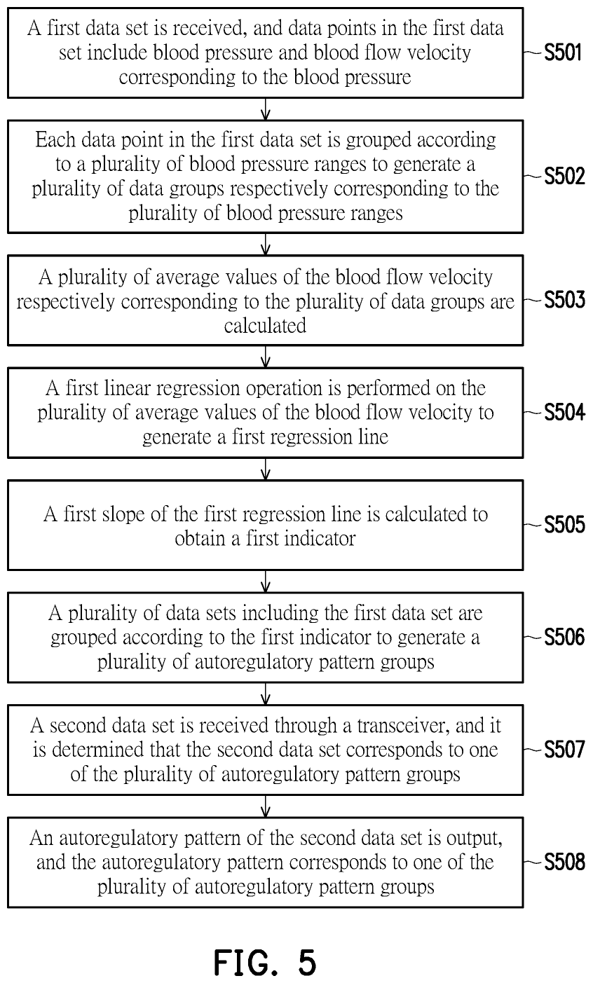

FIG. 5 is a flowchart of a method for evaluating an autoregulatory pattern of cerebral blood flow according to an embodiment of the disclosure. The method can be implemented by the electronic device 10 shown in FIG. 1. In step S501, a first data set is received, and data points in the first data set include blood pressure and blood flow velocity corresponding to the blood pressure. In step S502, each data point in the first data set is grouped according to a plurality of blood pressure ranges to generate a plurality of data groups respectively corresponding to the plurality of blood pressure ranges. In step S503, a plurality of average values of the blood flow velocity respectively corresponding to the plurality of data groups are calculated. In step S504, a first linear regression operation is performed on the plurality of average values of the blood flow velocity to generate a first regression line. In step S505, a first slope of the first regression line is calculated to obtain a first indicator. In step S506, a plurality of data sets including the first data set are grouped according to the first indicator to generate a plurality of autoregulatory pattern groups. In step S507, a second data set is received through a transceiver, and it is determined that the second data set corresponds to one of the plurality of autoregulatory pattern groups. In step S508, an autoregulatory pattern of the second data set is output, and the autoregulatory pattern corresponds to one of the plurality of autoregulatory pattern groups.

To sum up, the electronic device of the disclosure can perform the linear regression operation on the data sets including the data points of blood pressure and blood flow velocity according to parameters such as the blood flow velocity, the cerebrovascular resistance, and the blood pressure to generate the indicators of the data sets. The electronic device can group the data sets according to the indicators to determine the autoregulatory patterns of the data sets. Accordingly, the disclosure can evaluate an autoregulatory pattern of cerebral blood flow of a patient in a non-invasive manner.

What is claimed is:

1. An electronic device for evaluating an autoregulatory pattern of cerebral blood flow, comprising:
a transceiver; and
a processor, coupled to the transceiver, and configured to:
receive a first data set through the transceiver, wherein a data point in the first data set comprises blood pressure and blood flow velocity corresponding to the blood pressure;
group each data point in the first data set according to a plurality of blood pressure ranges to generate a plurality of data groups respectively corresponding to the plurality of blood pressure ranges;
calculate a plurality of average values of the blood flow velocity respectively corresponding to the plurality of data groups;
perform a first linear regression operation on the plurality of average values of the blood flow velocity to generate a first regression line;
calculate a first slope of the first regression line to obtain a first indicator;
group a plurality of data sets comprising the first data set according to the first indicator to generate a plurality of autoregulatory pattern groups;
receive a second data set through the transceiver, and determine that the second data set corresponds to one of the plurality of autoregulatory pattern groups; and
output an autoregulatory pattern of the second data set, wherein the autoregulatory pattern corresponds to one of the plurality of autoregulatory pattern groups.

2. The electronic device according to claim 1, wherein the processor is further configured to:
calculate cerebrovascular resistance based on the blood pressure and the blood flow velocity;
calculate a plurality of average values of the cerebrovascular resistance respectively corresponding to the plurality of data groups;
perform a second linear regression operation on the plurality of average values of the cerebrovascular resistance to generate a second regression line;
calculate a second slope of the second regression line to obtain a second indicator; and
group the plurality of data sets according to the first indicator and the second indicator to generate the plurality of autoregulatory pattern groups.

3. The electronic device according to claim 1, wherein the processor is further configured to:
calculate a plurality of standard deviations of the blood flow velocity respectively corresponding to the plurality of average values of the blood flow velocity;
generate an image based on the plurality of average values of the blood flow velocity and the plurality of standard deviations of the blood flow velocity; and
output the image.

4. The electronic device according to claim 1, wherein the processor is further configured to:
communicatively connect to an ultrasonic instrument through the transceiver, and receive the blood flow velocity of the data point from the ultrasonic instrument.

5. The electronic device according to claim 1, wherein the processor is further configured to:
communicatively connect to a sphygmomanometer through the transceiver, and receive the blood pressure of the data point from the sphygmomanometer.

6. The electronic device according to claim 1, wherein each data point in the first data set corresponds to each heartbeat of a subject.

7. The electronic device according to claim 1, wherein the processor is further configured to:
perform an interpolation operation on the first data set to up-sample the first data set.

8. The electronic device according to claim 1, wherein the processor is further configured to:
output an alert message, in response to determining that the second data set corresponds to one of the plurality of autoregulatory pattern groups.

9. A method for evaluating an autoregulatory pattern of cerebral blood flow, comprising:
receiving a first data set, wherein a data point in the first data set comprises blood pressure and blood flow velocity corresponding to the blood pressure;
grouping each data point in the first data set according to a plurality of blood pressure ranges to generate a plurality of data groups respectively corresponding to the plurality of blood pressure ranges;
calculating a plurality of average values of the blood flow velocity respectively corresponding to the plurality of data groups;
performing a first linear regression operation on the plurality of average values of the blood flow velocity to generate a first regression line;
calculating a first slope of the first regression line to obtain a first indicator;
grouping a plurality of data sets comprising the first data set according to the first indicator to generate a plurality of autoregulatory pattern groups;
receiving a second data set, and determining that the second data set corresponds to one of the plurality of autoregulatory pattern groups; and
outputting an autoregulatory pattern of the second data set, wherein the autoregulatory pattern corresponds to one of the plurality of autoregulatory pattern groups.
